(19) United States
(12) Patent Application Publication
(10) Pub. No.: US 2018/0146859 A1
(43) Pub. Date: May 31, 2018

(54) INFORMATION ACQUISITION APPARATUS

(71) Applicant: CANON KABUSHIKI KAISHA, Tokyo (JP)

(72) Inventors: Kenji Oyama, Tokyo (JP); Toshinobu Tokita, Yokohama-shi (JP); Takaaki Nakabayashi, Kawasaki-shi (JP)

(21) Appl. No.: 15/816,739

(22) Filed: Nov. 17, 2017

(30) Foreign Application Priority Data
Nov. 25, 2016 (JP) 2016-229313

(51) Int. Cl. A61B 5/00; A61B 8/00

(52) U.S. Cl. CPC A61B 5/0095; A61B 8/463; A61B 8/4444

(57) ABSTRACT

An information acquisition apparatus includes a light emission unit configured to emit light to irradiate a subject with the light, and an ultrasonic probe configured to receive an ultrasonic wave generated from irradiation of the subject with the light to output an electric signal wherein the light emission unit is provided in such a manner that a surface of the light emission unit in contact with the subject protrudes more toward a side of the subject than a surface of the ultrasonic probe in contact with the subject, and wherein an emission direction of the light emitted from the light emission unit is inclined in a direction in which the emission direction is away with respect to a line perpendicular to the surface of the ultrasonic probe where the ultrasonic probe is in contact with the subject.

### BACKGROUND

### Field of the Disclosure

The present disclosure relates to an information acquisition apparatus.

### Description of the Related Art

As a method for specifically imaging angiogenesis caused due to cancer, photoacoustic imaging (hereinafter referred to as Photoacoustic Imaging (PAI)) has been attracting attention. The PAI is a method that irradiates a subject with irradiation light (near-infrared light), and receives a photoacoustic wave generated from inside the subject with use of an ultrasonic probe to generate an image. FIGS. 7A and 7B schematically illustrate a hand-held photoacoustic imaging apparatus discussed in Japanese Patent Application Laid-Open No. 2016-49212. Referring to FIGS. 7A and 7B, an acoustic wave generated from skin of a subject propagates as indicated by dotted lines U′ as propagation illustration. Clutter noise N1 is reflected or scattered on, for example, an interface 1001 inside a subject 1000. The subject 1000 is irradiated with irradiation light L2.

In FIG. 7A, an ultrasonic probe 701 receives a photoacoustic signal. The photoacoustic imaging apparatus generates a photoacoustic image based on the photoacoustic signal received by the ultrasonic probe 701 with use of a processing unit (not illustrated). A light irradiation unit 703 irradiates the subject 1000 with the light L2. The photoacoustic imaging apparatus is configured to be able to change a direction in which the light irradiation unit 703 irradiates the subject 1000 with the light L2 by an irradiation direction changeable unit (not illustrated). Japanese Patent Application Laid-Open No. 2016-49212 and Japanese Patent Application Laid-Open No. 2016-49214 illustrate the photoacoustic imaging apparatus in such a manner that an optical axis of the light irradiation unit 703 is oriented toward a sound axis of a detection unit, i.e., the ultrasonic probe 701.

Japanese Patent Application Laid-Open No. 2016-49212 discusses such a configuration that the light irradiation unit 703 is oriented toward the ultrasonic probe 701. In such a configuration, as illustrated in FIG. 7B, the skin, which is a surface of the subject 1000, is irradiated with the light L2 emitted from the light irradiation unit 703, and the photoacoustic wave (an ultrasonic wave) is generated from the skin. Then, this photoacoustic wave generated from the skin propagates through the subject 1000 as indicated by the dotted lines U′ in FIG. 7B. This propagating photoacoustic wave is undesirably received by the ultrasonic probe 701 as indicated by arrows N1 in FIG. 7B via a countless number of interfaces 1001 and scatterers existing in the subject 1000, such as an interface 1001 between fat and a fascia. This noise undesirably received under such a circumstance is called clutter noise. The reception of the clutter noise leads to undesirable deterioration in a ratio (a signal-to-noise (SN) ratio) of noise to a signal derived from an absorber, such as blood, in the subject 1000, and a contrast ratio of a generated image.

### SUMMARY

The present disclosure is directed to reducing the clutter noise to increase the ratio of the noise to the signal derived from the absorber in the subject.

According to an aspect of the present invention, an information acquisition apparatus includes a light emission unit configured to emit light to irradiate a subject with the light, an ultrasonic probe configured to receive an ultrasonic wave generated from irradiation of the subject with the light to output an electric signal, and an information acquisition unit configured to acquire information about the subject from the electric signal, wherein the light emission unit is provided in such a manner that a surface of the light emission unit in contact with the subject protrudes more toward a side of the subject than a surface of the ultrasonic probe in contact with the subject, and wherein an emission direction of the light emitted from the light emission unit is inclined in a direction in which the emission direction is away with respect to a line perpendicular to the surface of the ultrasonic probe where the ultrasonic probe is in contact with the subject.

According to another aspect of the present invention, an information acquisition apparatus includes a light emission unit configured to emit light to irradiate a subject with the light, an ultrasonic probe configured to receive a photoacoustic wave generated from irradiation of the subject with the light and convert the received photoacoustic wave into an electric signal, and a processing unit configured to generate a photoacoustic image from the electric signal, wherein the light emission unit includes an emission end surface that is in contact with the subject, and wherein the emission end surface is arranged in such a manner that an emission direction of light emitted from the emission end surface is inclined so as to be separated away relative to a sound axis of the ultrasonic probe.

Further features of the present invention will become apparent from the following description of exemplary embodiments with reference to the attached drawings.

### BRIEF DESCRIPTION OF THE DRAWINGS

FIG. 1 illustrates an entire configuration of an information acquisition apparatus according to an exemplary embodiment of the present invention.

FIGS. 2A, 2B, and 2C illustrate a configuration of an irradiation angle changeable unit according to the exemplary embodiment of the present invention.

FIGS. 3A, 3B, 3C, 3D, 3E, and 3F are each a perspective view illustrating an ultrasonic probe according to the exemplary embodiment of the present invention.

FIG. 4 illustrates irradiation angle change control according to a first exemplary embodiment of the present invention.

FIGS. 5A, 5B, and 5C illustrate condition setting regarding the irradiation angle according to a second exemplary embodiment and a third exemplary embodiment of the present invention.

FIGS. 6A and 6B illustrate a correction of a light distribution according to a fourth exemplary embodiment of the present invention.

FIGS. 7A and 7B illustrate a configuration of a conventional photoacoustic imaging apparatus.

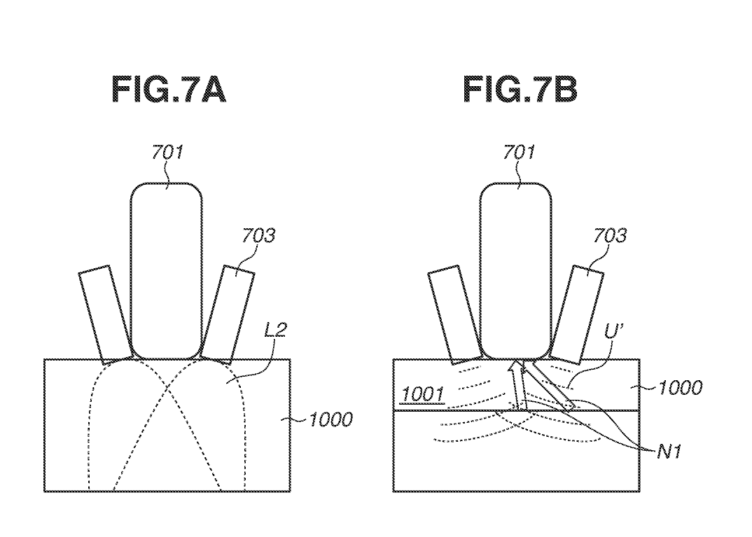

### DESCRIPTION OF THE EMBODIMENTS

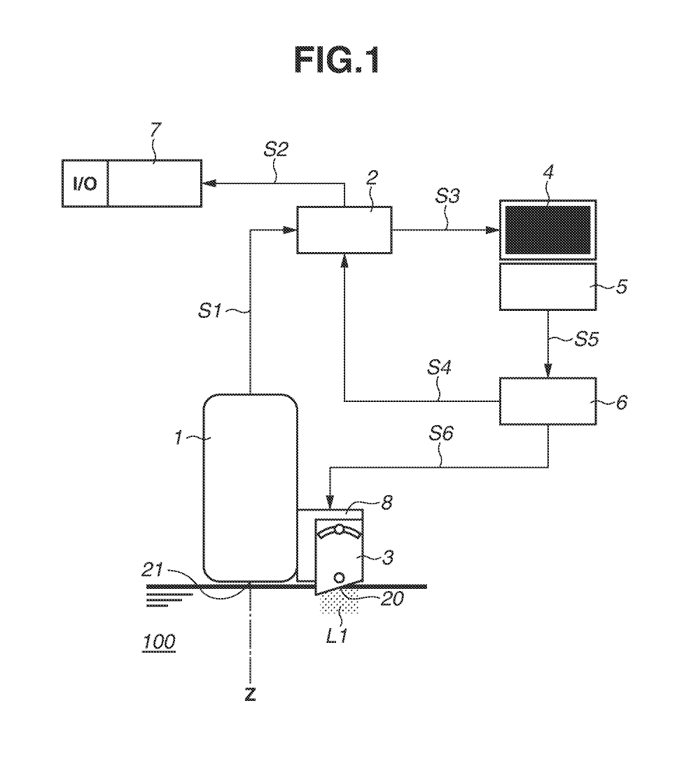

An overview of an information acquisition apparatus according to an exemplary embodiment of the present invention will be described with reference to FIG. 1.

The information acquisition apparatus according to the present exemplary embodiment includes a light emission unit 3 that emits light L1 to irradiate a subject 100 with the light L1, and an ultrasonic probe 1 that receives an ultrasonic wave (which can also be restated as an acoustic wave or a photoacoustic wave) generated from the irradiation of the subject 100 with the light (L1) to output an electric signal. Further, the information acquisition apparatus according to the present exemplary embodiment includes an information acquisition unit 2 that acquires information about the subject 100 from the acquired electric signal. The light emission unit 3 has a surface (a second surface) 20 in contact with a side of the subject 100, while the ultrasonic probe 1 also has a surface (a first surface) 21 in contact with the side of the subject 100. The surface 21 protrudes more toward the subject 100 than the surface 20. Further, an emission direction of the light emitted from the light emission unit is inclined in a direction in which the emission direction is away with respect to a line perpendicular to the first surface 21. Here, the emission direction of the light is a direction in which a line perpendicular to the second surface 20 extends to the subject 100. This inclination is not limited thereto as long as the line perpendicular to the second surface 20 is inclined relative to the line perpendicular to the first surface 21.

Due to such a configuration, the light L1 emitted from the light emission unit 3 to the subject 100 is oriented toward a direction away from the ultrasonic probe 1, so that an ultrasonic wave generated around a surface of the subject 100 is likely to proceed in a direction away from a sound axis z of the ultrasonic probe 1. As a result, the ultrasonic wave received by the ultrasonic probe 1 becomes less likely to contain the clutter noise, so that the information acquisition apparatus can acquire a high-contrast image when an image of the subject 100 is generated.

Further, the information acquisition apparatus according to the present exemplary embodiment may include an angle changeable unit (irradiation angle changeable unit) 8, which is configured to be able to change an angle defined between the line perpendicular to the first surface 21 and the line perpendicular to the second surface 20, and a control unit 6, which controls this angle. By such a configuration, the information acquisition apparatus according to the present exemplary embodiment can adjust the angle according to a shape of the subject 100 and an imaging target site. Further, as will be described below, the information acquisition apparatus according to the present exemplary embodiment can calculate such an optimum irradiation angle that the image of the subject 100 has high contrast, and acquire the image of the subject 100 at this angle.

Further, the information acquisition apparatus according to the present exemplary embodiment can include a processing unit 2, which calculates a light amount distribution inside the subject 100 and a quantitative analytic value inside the subject 100 according to the above-described angle. The light amount distribution inside the subject 100 varies according to the angle, so that this configuration allows the information acquisition apparatus to perform correction processing according thereto. In FIG. 1, the information acquisition unit and the processing unit are illustrated as the same block (2), but may be different units from each other (the same also applies hereinafter).

The control unit 6 is configured to control the angle changeable unit 8 based on at least a reference table recording a relationship between the target site in the subject 100 from which the information is acquired (a site to be imaged), and the angle. The reference table may be stored in a memory (not illustrated). Alternatively, in a case where the control unit 6 itself includes a built-in memory, the reference table may be stored in the built-in memory.

Further, displaying the above-described angle on a display unit (monitor) 4 allows a user to perform an operation of acquiring the information of the subject 100 while recognizing the light irradiation angle. The display unit 4 may include a display control unit that controls the display unit 4 (not illustrated). The display control unit may be built in the display unit 4 or may be provided as a different unit from the display unit 4.

An angle changeable mechanism of the angle changeable unit 8 is not especially limited as long as this mechanism is configured to be able to change the angle, and, for example, an actuator can be used therefor. Further, the ultrasonic probe 1 may be contained in a casing, and may be provided with a cover protecting a surface of the ultrasonic probe 1 on the subject side.

Further, in the information acquisition apparatus according to the present exemplary embodiment, the light emission unit 3 may include a contact detection sensor capable of acquiring information about a contact state between the light emission unit 3 and the subject 100. Now, the information about the contact state refers to a concept containing not only whether the light emission unit 3 is in contact with the subject 100 but also how much the light emission unit 3 is in contact with the subject 100. Then, the information acquisition apparatus according to the present exemplary embodiment may be configured to be able to switch whether to acquire the information about the subject 100 based on at least the information about the contact state. For example, the information acquisition apparatus according to the present exemplary embodiment can perform control such as refraining from acquiring the information about the subject 100 or refraining from irradiating the subject 100 with the light L1 if the contact is determined to be insufficient. Alternatively, the information acquisition apparatus according to the present exemplary embodiment may also acquire information about noise in advance in addition to acquiring the information about the subject 100, and display an alert indicating that the information about the subject 100 may contain a large amount of noise on the display unit 4.

The information acquisition apparatus according to the present exemplary embodiment may include a reception unit 5, which receives an input for performing the operation for acquiring the information about the subject 100 from the user. The reception unit 5 may also serve as an input device. When the reception unit 5 receives information about the target site in the subject 100 from which the information is acquired, the control unit 6 can perform control of executing a process of referring to the above-described reference table, a process of controlling the above-described angle, and a process of acquiring the information about the subject 100.

Further, when the reception unit 5 receives the information about the target site in the subject 100 from which the information is acquired, the control unit 6 may perform control of carrying out condition setting imaging for acquiring the information about the subject 100 while changing the above-described angle within a range where this angle is changeable. Further, the control unit 6 may perform control of displaying the angle during the condition setting imaging on the display unit 4.

Further, when the reception unit 5 receives information about the above-described angle, the control unit 6 can control the angle changeable unit 8 so that the subject 100 is irradiated with the light L1 at that angle, and acquire the information about the subject 100 at that angle.

Further, the information acquisition apparatus according to the present exemplary embodiment may include an angle calculation unit (not illustrated). The angle calculation unit may be configured to calculate such an angle that allows the region of interest to have highest contrast. Based on that, the control unit 6 can control the angle changeable unit 8 in such a manner that the subject 100 is irradiated with the light L1 at the calculated angle, and the ultrasonic probe 1 can receive the ultrasonic wave generated from the irradiation of the subject 100 with the light L1 at this angle.

In the following description, the exemplary embodiment of the present invention will be described in detail. The illustration of FIGS. 1 to 5 includes an acquired signal S1, subject information and various kinds of imaging conditions S2, image information S3, various kinds of imaging conditions S4 (including the irradiation angle information), a region of interest (ROI) setting and photoacoustic imaging S5, a driving control signal S6, condition setting imaging and photoacoustic imaging S7, and irradiation angle information S8. Further, the illustration of FIGS. 1 to 5 includes an ROI setting R, the irradiation light L1, the sound axis z, a sound axis surface p, and illustration U1 of the propagation of the ultrasonic wave (the acoustic wave) generated from the skin of the subject 100. The information acquisition apparatus according to the present exemplary embodiment is characterized in that the light emission unit 3 includes an emission end surface in contact with the subject 100, and this emission end surface is arranged so as to be inclined in such a manner that an emission direction of the light L1 emitted from the emission end surface is separated away relative to the sound axis z of the ultrasonic probe 1. The term “emission direction of the light” used here refers to a direction of a central axis of the light where, typically, a light intensity is maximized.

Further, as will be described below, the information acquisition apparatus according to the present exemplary embodiment may include an angle changeable unit that changes the emission direction of the light L1 by changing an angle of the emission end surface (which can also be referred to as an irradiation angle changeable unit). Further, the light emission unit 3 may be provided with a contact detection sensor that detects the contact state between the light emission unit 3 and the subject 100. In the following description, details thereof will be described.

FIG. 1 schematically illustrates the information acquisition apparatus (a PAI apparatus) according to the exemplary embodiment of the present invention. In FIG. 1, the ultrasonic probe 1 functions to receive the ultrasonic wave (which can also be restated as the acoustic wave or the photoacoustic wave) to convert it into the electric signal. Further, the ultrasonic probe 1 can also transmit an ultrasonic wave to the subject 100 and receive an ultrasonic wave reflected from inside the subject 100. Further, a surface of the ultrasonic probe 1 that transmits and receives the ultrasonic wave is in acoustic contact with the subject 100 via a not-illustrated acoustic matching agent (for example, sonar gel or water). The processing unit 2 functions to generate the image through an amplification, an analog/digital (A/D) conversion, and filter processing of the photoacoustic signal or the ultrasonic signal received by the ultrasonic probe 1. Further, the processing unit 2 can carry out beamforming when the ultrasonic probe 1 transmits and receives the ultrasonic wave. The light source (emission end) 3 includes the emission end surface that is in contact with the subject 100 and that emits the irradiation light L1, and irradiates the subject 100 with the irradiation light L1. In the present exemplary embodiment, the light emission unit 3 may be configured to be able to irradiate the subject 100 with light generated by a light source provided outside the information acquisition apparatus, or may be configured to include a light source. Accordingly, hereinafter, the “light emission unit” may be referred to as the “light source (emission end)”. The subject 100 is irradiated with the light L1 from the emission end surface 20 of the light emission unit 3.

In a case where a solid-state laser, such as a neodymium-doped yttrium aluminum garnet (Nd:YAG) laser, a titanium sapphire (Ti:sa) laser, an optical parametric oscillator (OPO) laser, and an alexandrite laser, is used as the light source generating the light, the light is transmitted to the light emission unit 3 via a bundle fiber (not illustrated). The transmission of the light is not limited to the method using the bundle fiber, and may be achieved with use of another light transmission method, such as light transmission using a reflective optical system via a prism or a mirror. Further, the light source is not limited to the solid-state laser and may be constructed with use of a laser diode (LD), a light emitting diode (LED), or the like, and may be configured to be included in the light emission unit 3. The light emission unit 3 is configured to be able to irradiate the subject 100 with, for example, pulsed light of approximately several nanoseconds to several hundred nanoseconds to generate the photoacoustic signal. The pulsed light can have a rectangular shape, but a Gaussian-shaped pulse is also usable. The monitor 4 functions to display image information generated by the processing unit 2 thereon. The input device 5 functions to allow the user to set imaging conditions for acquiring the photoacoustic or ultrasonic image. For example, a pointing device, such as a mouse, a trackball, and a touch panel, is used as the input device 5. The control unit 6 performs various kinds of control based on the imaging conditions input via the input device 5. Further, the control unit 6 reflects these imaging conditions in the processing unit 2. For example, when the acquisition of the photoacoustic image is started by an operation on the input device 5, the processing unit 2 stops the transmission of the ultrasonic wave, and causes the light emission unit 3 to emit the irradiation light L1 therefrom. Further, when acquiring the ultrasonic image, the user operates a selection of an imaging mode such as B-mode tomography, color Doppler, and power Doppler, a focus setting inside the subject 100, and the like with use of the input device 5. Then, according to this operation, the processing unit 2 carries out the beamforming to cause the ultrasonic probe 1 to transmit and receive the ultrasonic wave, and generates the image. A recording unit 7 records the subject information generated by the processing unit 2 and the various kinds of imaging conditions. Further, the information acquisition apparatus according to the present exemplary embodiment can transfer the subject information and the various kinds of imaging conditions from the recording unit 7 to a computer storing imaging data that is connected via a network or an external recording apparatus (not illustrated) such as a memory and a hard disk via an input/output I/O.

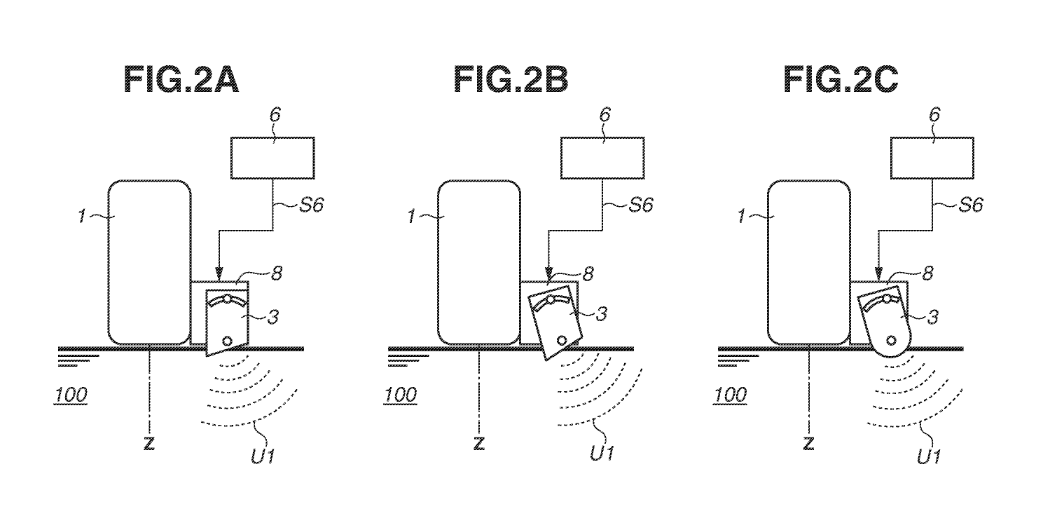

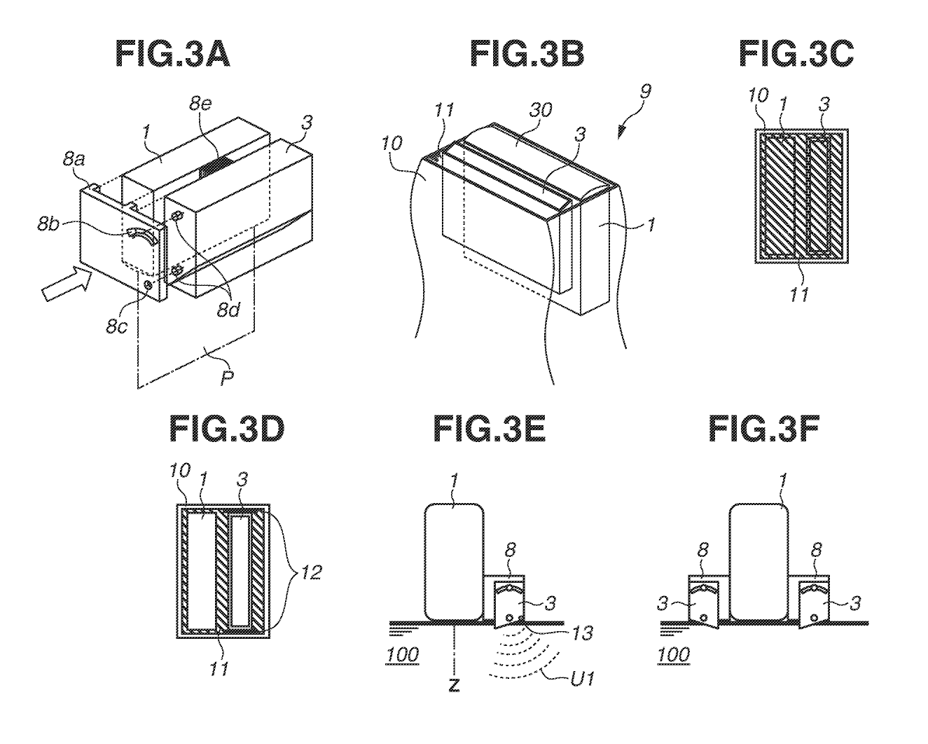

In the above-described photoacoustic imaging apparatus, the emission end surface where the light emission unit 3 is in contact with the subject 100 is inclined in the direction away from the sound axis z of the ultrasonic probe 1. Arranging the emission end surface in this manner corrects the skin, which is the surface of the subject 100, in the direction away from the sound axis z of the ultrasonic probe 1 as illustrated in FIG. 2A, and allows the acoustic wave generated from the skin to propagate in this direction, thereby succeeding in reducing the clutter noise that would be undesirably received via an interface and a scatterer inside the subject 100. As a result, a contrast ratio can be improved when an image of the imaging target is generated. All of FIGS. 1, and 2A, 2B, and 2C are side views, and views from a direction indicated by an arrow in FIG. 3A.

An intensity of the photoacoustic wave on the surface of the skin, which serves as a source of generating the clutter noise, and a distribution of the interface and the scatterer inside the subject 100, which serves as a source of reflecting the clutter noise, are different depending on a site in the subject 100, an individual variation, and/or the like. In other words, it is desirable to change the contact angle between the light source (emission end) 3 and the subject 100 according to the subject 100. Therefore, in FIG. 1, the irradiation angle changeable unit 8 makes the angle of the light source (emission end) 3 changeable based on driving control by the control unit 6. By being configured in this manner, the image acquisition apparatus according to the present exemplary embodiment can adjust the inclination angle of the emission end surface where the light source 3 is in contact with the subject 100. FIGS. 2A and 2B illustrate this angle changeability.

An equivalent scattering coefficient of the subject 100 is approximately 1 mm. Therefore, as far as a depth of the subject 100 reaches approximately 1 mm, the irradiation light L1 is scattered forward and therefore the image acquisition apparatus is affected by the angle at which the irradiation light L1 is incident. However, at a depth greater than that, the irradiation light L1 is scattered isotropically and therefore the image acquisition apparatus is less affected by the angle at which the irradiation light L1 is incident. In other words, the depth of the subject 100 that reaches or exceeds 1 mm can reduce the clutter noise while reducing the influence of the angle at which the irradiation light L1 is incident, thereby improving the contrast of the imaging target.

Further, the emission end surface of the light source 3 is illustrated as a flat surface, but is not limited thereto. The emission end surface of the light source 3 may have a curved surface and/or a stepped shape as long as the acoustic wave generated from the skin propagates in the direction away from the sound axis z. For example, in a case where the emission end surface of the light source 3 is a spherical surface as illustrated in FIG. 2C, even if a part of the acoustic wave generated from the skin also propagates in a direction toward the sound axis z of the ultrasonic probe 1, most of the acoustic wave propagates in the direction away from the sound axis z, so that the effect of reducing the clutter noise can be acquired.

Next, the irradiation angle changeable unit 8 will be described with reference to FIG. 3A. In FIG. 3A, the irradiation angle changeable unit 8 includes a plate 8a, which holds the ultrasonic probe 1 and further holds the light source 3 with respect to a direction other than a direction in which the angle thereof is changeable, and the plate 8a includes a guide hole 8b and a rotational center hole 8c. Two pins 8d are provided to the light source (emission end) 3, and one and the other of the pins 8d are fitted in the guide hole 8b and the rotational center hole 8c, respectively. FIG. 3A illustrates the irradiation angle changeable unit 8 in a state before the pins 8d are fitted in the holes. Then, the irradiation angle changeable unit 8 includes an actuator 8e for controlling the angle of the light source 3 with respect to the ultrasonic probe 1. Various types of motors, a piezoelectric element, a hydraulic/pneumatic/hydro-pneumatic cylinder, or the like can be employed as the actuator 8e. Further, the irradiation angle changeable unit 8 can also control positioning by using not only the actuator 8e but also a spring (not illustrated). According to the above-described configuration, the irradiation angle changeable unit 8 allows the image acquisition apparatus to change the angle of the light source 3 along the guide hole 8b around the rotational center hole 8c. In other words, the image acquisition apparatus can adjust the angle at which the acoustic wave generated from the skin is separated away from the sound axis z (the states illustrated in FIGS. 2A and 2B), and reduce the clutter noise according to the distribution of the interface and the scatterer inside the subject 100.

The irradiation angle changeable unit 8 is not limited to the configuration described with reference to FIG. 3A as long as the irradiation angle changeable unit 8 is configured to be at least able to change the angle of the light source 3 with respect to the ultrasonic probe 1 with use of the actuator 8e. For example, the irradiation angle changeable unit 8 may also be constructed by applying a link mechanism.

FIG. 3B illustrates an external appearance of a photoacoustic probe 9 when the reception surface of the ultrasonic probe 1 and the emission end surface of the light source (emission end) 3 are laid face up. In FIG. 3B, the photoacoustic probe 9 includes a casing 10. The casing is illustrated as having a corner and a ridge, but, actually, the corner and the ridge thereof is desirably tapered and rounded and the casing 10 is also required to have a curved or dented shape so as to allow an operator to easily hold it especially in the case of the hand-held apparatus. The photoacoustic probe 9 includes a cover 11, and thin resin, such as polyethylene terephthalate (PET) and urethane rubber, is attached to a subject side of the casing 10. The provision of this cover 11 can prohibit the acoustic matching agent such as sonar gel and water from entering the casing 10, thereby preventing or reducing a breakage of the actuator 8e (not illustrated) inside the photoacoustic probe 9. As the cover 11, the thin resin, such as PET and urethane rubber, is attached to the subject side of the casing 10.

FIG. 3C illustrates the photoacoustic probe 9 illustrated in FIG. 3B as viewed from the subject side. FIGS. 3B and 3C illustrate the cover 11 (a hatched portion in FIG. 3C) provided so as to cover the ultrasonic probe 1 and the emission end surface of the light source 3 altogether. However, the cover 11 is not limited thereto, and may be provided so as to expose the reception surface of the ultrasonic probe 1 from the photoacoustic probe 9 as illustrated in FIG. 3D. Further, the emission end surface of the light source 3 may also be exposed from the photoacoustic probe 9. Since the angle of the light source 3 is changeable, a seal member 12 is provided at a portion where the light source 3 and the casing 10 are located close to each other, and the provision of the seal member 12 prevents the acoustic matching agent from entering the photoacoustic probe 9.

Further, a light reflective coating (a light reflective film) 30, such as chrome and gold, is provided on the reception surface of the ultrasonic probe 1 or a portion of the cover 11 that covers the reception surface in the case where the cover 11 is placed over this reception surface. The provision of this light reflective coating 30 can eliminate or reduce a photoacoustic wave generated when the scattering irradiation light hits the reception surface of the ultrasonic probe 1 and thus reduce a noise source, thereby further improving the contrast.

The light source (emission end) 3 may be unable to allow the acoustic wave generated from the skin to propagate in the direction away from the sound axis z of the ultrasonic probe 1 as described with reference to FIGS. 2A an 2B unless the light source (emission end) 3 is in contact with the subject 100 and capable of correcting the surface in the direction of the angle thereof. Therefore, in FIG. 3E, a contact detection sensor 13 is provided at a portion of the light source (emission end) 3 in contact with the subject 100. If the contact is determined to be insufficient by the contact detection sensor 13, the angle of the subject 100 in contact with the light source (emission end) 3 is interpreted as failing to be oriented in the direction sufficiently away from the sound axis z of the ultrasonic probe 1. Then, the image acquisition apparatus displays and/or records that the photoacoustic data is not acquired or is acquired but may contain a large amount of clutter noise. An optical sensor, a piezoelectric sensor, an electrostatic sensor, an ultrasonic sensor, a pressure-type sensor, or the like can be employed as the contact detection sensor 13. By being configured in this manner, the image acquisition apparatus can orient the direction of the surface of the subject 100 with which the light source (emission end) 3 is in contact in the direction away from the sound axis z of the ultrasonic probe 1, thereby acquiring an ultrasonic image in which the clutter noise is further reliably reduced.

Further, the image acquisition apparatus can ensure that the irradiation light L1 is incident on the subject 100 by the control of acquiring the photoacoustic data only when the contact is determined to be established by the contact detection sensor 13. Therefore, the image acquisition apparatus can reduce a release of the irradiation light L1 into the air, thereby improving safety.

The light source (emission end) 3 described so far is located on one side of the ultrasonic probe 1, but is not limited thereto and may be provided on both sides of the ultrasonic probe 1 as illustrated in FIG. 3F or may be provided so as to surround the ultrasonic probe 1 besides that.

Further, the image acquisition apparatus has been illustrated assuming that the ultrasonic probe 1 is constructed with use of a 1-dimensional (D) array transducer, but how elements are arrayed is not limited thereto. For example, the present exemplary embodiment is also applicable to a 1.5D array transducer and a 2D array transducer, and is further also applicable to a convex-type transducer, a sector-type transducer, a concave-type transducer, and the like.

In the following description, each exemplary embodiment will be described.

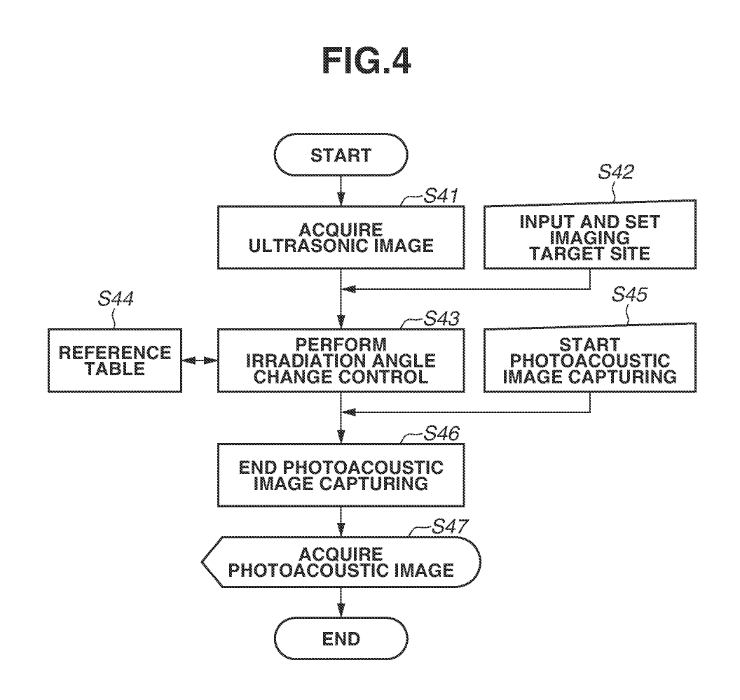

A first exemplary embodiment will be described as irradiation angle change control by the control unit 6 with reference to FIGS. 1 and 4. In FIG. 4, the change control includes the following processes.

In step S41, the ultrasonic image is acquired. Transmission beam subjected to the beamforming by the processing unit 2 is transmitted from the ultrasonic probe to inside the subject 100. Then, the ultrasonic wave reflected from inside the subject 100 is received by the ultrasonic probe 1, and the received signal is subjected to the amplification, the A/D conversion, and the filter processing by the processing unit 2, through which the ultrasonic image is generated and displayed on the monitor 4.

In step S42, the imaging target site is input and set. The operator views the ultrasonic image displayed in step S41 and sets a region to be processed as the region of interest with use of the input device 5.

In step S43, the irradiation angle change control is performed. The reference table (step S44) is referred to according to the imaging target site set in step S42. Then, the control unit 6 controls the driving of the irradiation angle changeable unit 8 to adjust the angle of the light source (emission end) 3 in contact with the subject 100.

Step S44 is the reference table. The angle of the light source (emission end) 3 in contact with the subject 100 for each imaging site is recorded in the reference table. The reference table is referred to in step S43. The reference table records the angle of the light source (emission end) 3 according to the imaging site (e.g., neck portion: 20 degrees, breast: 35 degrees, hand and finger: 10 degrees, and lower limb: 20 degrees). In the present exemplary embodiment, this angle is determined in consideration of not only a structure of the interface and the scatterer inside the imaging site but also hardness and softness. The angle of the light source (emission end) 3 in contact with the subject 100 is defined assuming that an angle in parallel with the subject 100 is 0 degree.

In step S45, the photoacoustic imaging operation is performed, and the operator performs the photoacoustic image capturing operation via the input device 5.

In step S46, the photoacoustic image capturing is stopped according to the operation in step S45, and the light source 3 emits the light L1 and the photoacoustic signal is received. In a case where the light L1 is emitted and the signal is received a plurality of times for acquiring the photoacoustic signal, the photoacoustic image capturing may be performed during a time period from the reception of the signal to the next emission of the light L1.

In step S47, a photoacoustic image is generated. The photoacoustic signal received by the ultrasonic probe 1 is subjected to the amplification, the A/D conversion, and the filter processing by the processing unit 2, through which the photoacoustic image is generated and displayed on the monitor 4. As a method for displaying the image on the monitor 4, the ultrasonic image acquired in step S41 and the photoacoustic image acquired in step S46 are displayed in color and in monochrome, respectively, while being superimposed on each other. The image displayed in color and the image displayed in monochrome may be reversed, or the images may also be displayed by a method that displays the images side by side or on top of each other without superimposing them or a method that displays the images while switching them.

According to the above-described method, the image acquisition apparatus allows the operator to determine the irradiation angle as soon as the operator sets the subject site.

The first exemplary embodiment has been described as the method that causes the operator to set the imaging site and causes the irradiation angle changeable unit 8 to adjust the angle of the light source (emission end) 3. A second exemplary embodiment will be described as a condition setting method that acquires a photoacoustic image while changing the irradiation angle within the changeable range and sets the angle to the irradiation position that can achieve a photoacoustic image desired by the operator.

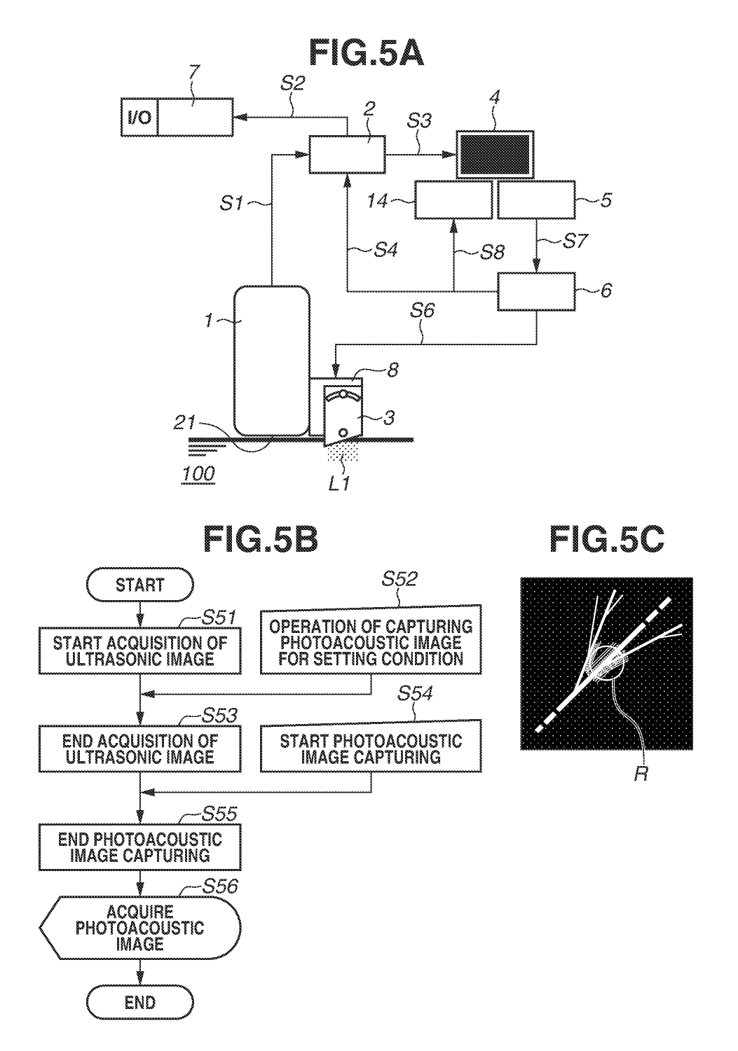

FIG. 5A illustrates the image acquisition apparatus illustrated in FIG. 1 that is additionally provided with a display unit 14 for presenting the irradiation angle. The irradiation position information may also be displayed on the monitor 4 without the display unit 14 provided.

Next, referring to FIG. 5B, a flow of determining the irradiation position includes the following processes.

In step S51, an ultrasonic image is acquired. The method for acquiring the ultrasonic image is similar to step S41 described with reference to FIG. 4 according to the first exemplary embodiment, and therefore a description thereof will be omitted here.

In step S52, an operation of capturing the photoacoustic image for setting the condition is performed. The operator operates condition setting imaging with use of the input device 5 illustrated in FIG. 5A.

In step S53, the acquisition of the ultrasonic image is stopped according to the operation in step S52, and the photoacoustic image is acquired while the angle of the irradiation light L1 is changed by the irradiation angle changeable unit 8. Further, the irradiation angle is displayed on the display unit 14. The operator can recognize an irradiation angle that allows the region of interest to have high contrast by viewing the photoacoustic image displayed on the monitor 4 and the irradiation angle displayed on the display unit 14. A method for acquiring the photoacoustic image is similar to step S46 and step S47 described with reference to FIG. 4 according to the first exemplary embodiment, and therefore a description thereof will be omitted here.

In step S54, the photoacoustic imaging operation is performed. The operator sets the irradiation angle with use of the input device 5 in such a manner that the irradiation angle matches the irradiation angle that allows the region of interest to have high contrast that has been recognized by the operator in step S53, and performs the photoacoustic image capturing operation.

In step S55, the photoacoustic image capturing is stopped according to the operation in step S54, and the light source 3 emits the light L1 and the photoacoustic signal is received. Details thereof are similar to step S46 described with reference to FIG. 4 according to the first exemplary embodiment, and therefore a description thereof will be omitted here.

In step S56, a photoacoustic image is generated, and the generated photoacoustic image is displayed on the monitor 4. Details thereof are similar to step S47 described with reference to FIG. 4 according to the first exemplary embodiment, and therefore a description thereof will be omitted here.

According to the above-described method, the image acquisition apparatus can acquire the photoacoustic image under the condition that can achieve a highly visible and high-contrast image for the operator by capturing the photoacoustic image for setting the condition.

Further, the present exemplary embodiment allows the operator to predict the irradiation angle that should be set based on the subject site and the ultrasonic image acquired in step S51, and reduce an angle by which the irradiation light L1 is moved in the condition setting operation in step S52, thereby reducing a time taken for the imaging. Further, the present exemplary embodiment allows the operator to set the angle in step S54 while omitting this condition setting operation in step S52 itself when the operator becomes skillful. In this manner, the present exemplary embodiment allows the operator to improve the operator's skill level through the condition setting imaging.

In the condition setting described so far, the operator recognizes the irradiation position that allows the region of interest to have high contrast, and sets the irradiation angle desired by the operator. On the other hand, condition setting that will be described in a third exemplary embodiment is a method that automatically sets the irradiation angle. FIG. 5B also serves as a drawing for the third exemplary embodiment, and therefore will be used assuming that “0” is added to each of the step numbers.

In step S510, an ultrasonic image is acquired.

In step S520, an operation of setting the region of interest and an operation of capturing the photoacoustic image for setting the condition are performed. The operator sets the region to be processed as the region of interest with use of the input device 5 by viewing the ultrasonic image displayed in step S510. After that, the operator operates the condition setting imaging with use of the input device 5.

In step S530, the acquisition of the ultrasonic image is stopped according to the operation in step S520, and the light source 3 emits the light L1 and the photoacoustic signal is received while the irradiation angle is adjusted by the irradiation angle changeable unit 8. Then, the processing unit 2 determines an irradiation position that allows the region of interest to have highest contrast, and causes the control unit 6 to drive the irradiation angle changeable unit 8.

In step S540, the photoacoustic imaging operation is performed. The operator performs the photoacoustic image capturing operation with use of the input device 5.

In Step S550, the photoacoustic image capturing is stopped according to the operation in step S540, and the photoacoustic image is acquired.

In step S560, a photoacoustic image is generated, and the generated photoacoustic image is displayed on the monitor 4.

To determine the irradiation position that allows the region of interest to have the highest contrast in step S530, the processing unit 2 first acquires, with respect to the set region of interest, a luminance value of the photoacoustic image in this region. Then, the processing unit 2 calculates the contrast as the contrast=a maximum value/an average value, and determines an irradiation position that allows the contrast to be maximized. For example, FIG. 5C schematically illustrates a part of the photoacoustic image, which is an image containing a mixture of the imaging target, noise, and an artifact. Then, the processing unit 2 acquires the luminance value in the region of interest when the region of interest is set for each voxel (in a case of a 3D display) or each pixel (in a case of a 2D display or in a case of a maximum intensity projection (MIP) at a predetermined depth of 3D). The luminance is expressed by 16 bits (65536 tones). The processing unit 2 acquires the contrast from this luminance. The contrast is acquired from the ratio of the maximum value and the average value in the region of interest, but is not limited thereto and can also be acquired with use of a method that separates the imaging target from the noise and the artifact in a further advanced manner with use of an image recognition technique and acquires the contrast from a ratio between them.

According to the above-described method, the image acquisition apparatus can acquire the photoacoustic image in which the region of interest has high contrast by capturing the photoacoustic image for setting the condition.

A fourth exemplary embodiment will be described as each light distribution correction according to the irradiation angle that is carried out by the processing unit 2.

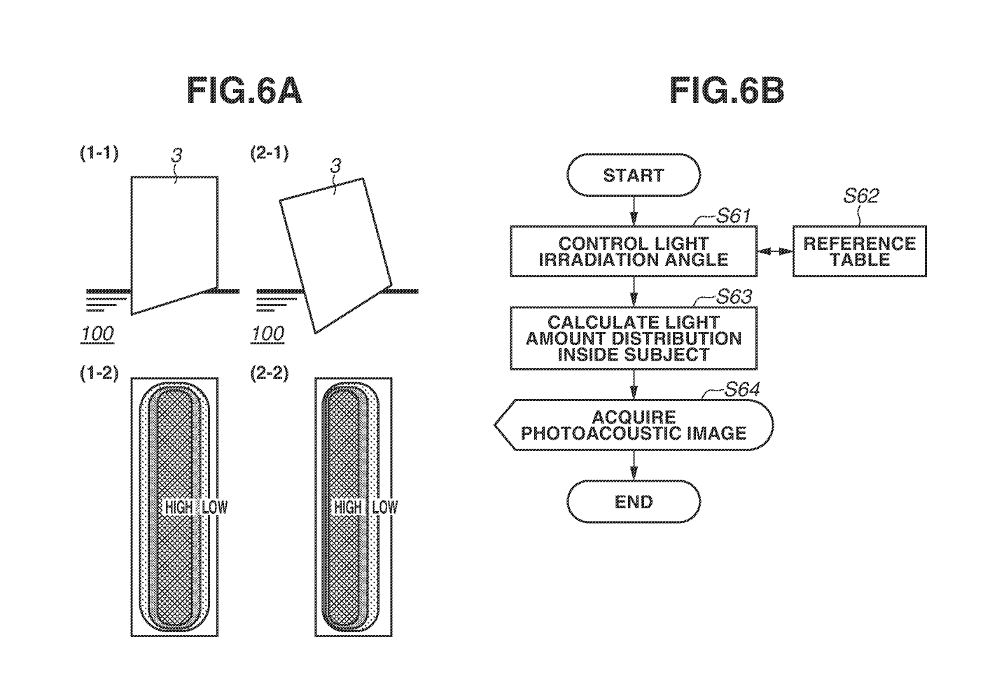

FIG. 6A schematically illustrates that the light amount distribution on the surface of the subject 100 varies according to the irradiation angle of the light source 3. A part (1-1) illustrates a state A of the light source 3, and a part (1-2) illustrates an example of a light amount distribution on the surface of the subject 100 in the state A.

Suppose that the light amount distribution on the surface of the subject 100 is vertically and horizontally symmetric when the irradiation angle of the light source 3 is in the state A. Then, when the irradiation angle of the light source 3 is changed into a state B, the light amount distribution on the surface of the subject 100 is distorted and undesirably loses the horizontal symmetry. In this case, the distribution of the amount of the light spreading inside the subject 100 undesirably varies. For example, an initial sound pressure of the photoacoustic signal is expressed as p=Γ·μa·φ. In this equation, Γ, μa, and φ represent a Grueneisen coefficient, an absorption coefficient, and a light amount, respectively. When the absorption coefficient is calculated from this equation, the absorption coefficient can be calculated by transforming the equation into μa=p/(Γ·φ), acquiring the initial sound pressure p by converting a received sound pressure into it, and using a known value as the Grueneisen coefficient Γ, as long as the light amount φ can be found out. Employable calculation methods therefor include a calculation using the thermal diffusion equation, the Monte Carlo method, and/or the like from an optical constant μeff of a tissue inside the subject 100, a total light amount of the irradiation light L1, the irradiation position, and the light amount distribution on the surface of the subject 100. These values are known if being measured in advance except for the irradiation position, and therefore the calculation can be realized by setting the irradiation position as a parameter. This calculation does not have to be carried out every time the irradiation angle is changed, and can be achieved by preparing the light amount distribution inside the subject 100 with respect to the irradiation position in the form of a table or a conversion equation in advance. By being configured in this manner, the information acquisition apparatus can figure out the light amount distribution inside the subject 100, thereby improving accuracy of calculating the absorption coefficient inside the subject 100. By being also applied when a quantitative analytic value, such as a total hemoglobin amount in blood and an oxygen saturation in blood, besides the absorption coefficient distribution inside the subject 100, is acquired, the present exemplary embodiment can improve accuracy of calculating them.

Next, a flow of correcting the light distribution will be described with reference to FIG. 6B. A part (2-1) illustrates a state B of the light source 3, and a part (2-2) illustrates an example of a light amount distribution on the surface of the subject 100 in the state B.

In step S61, the processing unit 2 controls the irradiation angle changeable unit 8 to adjust the angle at which the light source (emission end) 3 is in contact with the surface of the subject 100. Step S43 according to the first exemplary embodiment that has been described with reference to FIG. 4 and step S54 according to the second exemplary embodiment that has been described with reference to FIG. 5B correspond to this adjustment.

Step S62 is a reference table. The reference table stores the light amount distribution on the surface of the subject 100 according to the irradiation angle and a background optical constant (an absorption coefficient or a scattering coefficient) for each subject site.

In step S63, the processing unit 2 calculates the light amount distribution inside the subject 100 from the adjusted irradiation angle and the optical constant of the subject site. Then, the processing unit 2 calculates the absorption coefficient inside the subject 100 based on the photoacoustic signal received by the ultrasonic probe 1 (not illustrated). In the case where the oxygen saturation in blood is acquired, at least two-wavelength photoacoustic data should be used. In this case, respective photoacoustic signals having a wavelength λ1 and a wavelength λ2 are received at the stage of step S46 according to the first exemplary embodiment that has been described with reference to FIG. 4 or step S55 according to the second exemplary embodiment that has been described with reference to FIG. 5B.

In step S64, an ultrasonic image is generated. Because step S64 is similar to step S47 according to the first exemplary embodiment that has been described with reference to FIG. 4 or step S56 according to the second exemplary embodiment that has been described with reference to FIG. 5B, a description thereof will be omitted here.

According to the above-described method, the information acquisition apparatus can acquire the absorption coefficient of the photoacoustic image and the quantitative data of the oxygen saturation with further higher accuracy.

Since the emission end surface of the light emission unit 3 in contact with the subject 100 and the sound axis z of the ultrasonic probe 1 are inclined in the direction away relative to each other, the acoustic wave generated from around the surface of the subject 100 propagates away from the sound axis z of the ultrasonic probe 1, which can contribute to reducing the influence of the so-called clutter noise. As a result, the contrast of the imaging target can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-229313, filed Nov. 25, 2016, which is hereby incorporated by reference herein in its entirety.

What is claimed is:

1. An information acquisition apparatus comprising:
a light emission unit configured to emit light to irradiate a subject with the light;
an ultrasonic probe configured to receive an ultrasonic wave generated from irradiation of the subject with the light to output an electric signal; and
an information acquisition unit configured to acquire information about the subject from the electric signal,
wherein the light emission unit is provided in such a manner that a surface of the light emission unit in contact with the subject protrudes more toward a side of the subject than a surface of the ultrasonic probe in contact with the subject, and
wherein an emission direction of the light emitted from the light emission unit is inclined in a direction in which the emission direction is away with respect to a line perpendicular to the surface of the ultrasonic probe where the ultrasonic probe is in contact with the subject.

2. The information acquisition apparatus according to claim 1, further comprising:
an angle changeable unit operative to change an angle between the emission direction of the light and the line perpendicular to the surface of the ultrasonic probe where the ultrasonic probe is in contact with the subject; and
a control unit configured to control the angle.

3. The information acquisition apparatus according to claim 2, further comprising a processing unit configured to calculate a light amount distribution inside the subject and a quantitative analytic value inside the subject according to the angle.

4. The information acquisition apparatus according to claim 2, wherein the control unit is configured to control the angle changeable unit based on at least a reference table recording a relationship between a target site in the subject about which information is acquired and the angle.

5. The information acquisition apparatus according to claim 2, wherein control is performed so that the angle is displayed on a display unit.

6. The information acquisition apparatus according to claim 2, wherein the angle changeable unit includes at least an actuator.

7. The information acquisition apparatus according to claim 1, further comprising:
a casing incorporating the ultrasonic probe; and
a cover protecting the surface of the ultrasonic probe where the ultrasonic probe is in contact with the subject.

8. The information acquisition apparatus according to claim 1, wherein the light emission unit includes a contact detection sensor capable of acquiring information about a contact state between the light emission unit and the subject.

9. The information acquisition apparatus according to claim 8, wherein whether to acquire the information about the subject is switched based on at least the information about the contact state that is acquired by the contact detection sensor.

10. The information acquisition apparatus according to claim 8, wherein the information acquisition unit is configured to acquire information about noise in the information about the subject based on at least the information about the contact state that is acquired by the contact detection sensor.

11. The information acquisition apparatus according to claim 1, further comprising a display control unit configured to cause a display unit to display the information about the subject.

12. The information acquisition apparatus according to claim 1, further comprising a reception unit configured to receive from a user an input for performing an operation for acquiring the information about the subject.

13. The information acquisition apparatus according to claim 4, further comprising a reception unit configured to receive from a user an input for performing an operation for acquiring the information about the subject,
wherein, when the reception unit receives information about the target site in the subject about which the information is acquired, the control unit performs control for
referring to the reference table,
controlling the angle, and
acquiring the information about the subject.

14. The information acquisition apparatus according to claim 2, further comprising a reception unit configured to receive from a user an input for performing an operation for acquiring the information about the subject,
wherein, when the reception unit receives information about a target site in the subject about which information is acquired, the control unit performs control for carrying out condition setting imaging for acquiring the information about the subject while changing the angle within a range where the angle is changeable.

15. The information acquisition apparatus according to claim 14, wherein the control unit performs control for displaying the angle during the condition setting imaging on a display unit.

16. The information acquisition apparatus according to claim 2, further comprising a reception unit configured to receive from a user an input for performing an operation for acquiring the information about the subject,
wherein, when the reception unit receives information about the angle, the control unit controls the angle changeable unit so that the subject is irradiated with the light at the angle, and the control unit performs control for acquiring the information about the subject at the angle.

17. The information acquisition apparatus according to claim 2, further comprising:
a reception unit configured to receive from a user an input for performing an operation for acquiring the information about the subject; and
an angle calculation unit configured to calculate the angle,
wherein, when the reception unit receives information about a region of interest in the subject,
the angle calculation unit calculates the angle that allows the region of interest to have highest contrast,
the control unit controls the angle changeable unit so that the subject is irradiated with the light at the angle, and
the ultrasonic probe receives ultrasonic wave generated from the irradiation of the subject with the light at the angle.

18. The information acquisition apparatus according to claim 2, further comprising a processing unit configured to perform
controlling the angle,
calculating a light amount distribution inside the subject based on the angle and an optical constant inside the subject, and
carrying out a quantitative analysis inside the subject.

19. An information acquisition apparatus comprising:
a light emission unit configured to emit light to irradiate a subject with the light;
an ultrasonic probe configured to receive a photoacoustic wave generated from irradiation of the subject with the light and convert the received photoacoustic wave into an electric signal; and
a processing unit configured to generate a photoacoustic image from the electric signal,
wherein the light emission unit includes an emission end surface that is in contact with the subject, and
wherein the emission end surface is arranged in such a manner that an emission direction of light emitted from the emission end surface is inclined so as to be separated away relative to a sound axis of the ultrasonic probe.

20. The information acquisition apparatus according to claim 19, further comprising an angle changeable unit configured to change the emission direction by changing an angle of the emission end surface.

21. The information acquisition apparatus according to claim 19, wherein a contact detection sensor configured to detect a contact state between the light emission unit and the subject is provided to the light emission unit.

22. The information acquisition apparatus according to claim 19, further comprising a display control unit configured to control display of the photoacoustic image.

23. The information acquisition apparatus according to claim 19, further comprising a display unit configured to display the photoacoustic image.

24. The information acquisition apparatus according to claim 19, further comprising an input device for performing an operation of capturing the photoacoustic image.
